(19) United States
(12) Patent Application Publication
(10) Pub. No.: US 2025/0120837 A1
(43) Pub. Date: Apr. 17, 2025

(54) Dynamic Splint

(71) Applicant: Kenai Design, LLC, Newtown, CT (US)

(72) Inventor: Eric Kolb, Newtown, CT (US)

(21) Appl. No.: 18/685,267

(22) PCT Filed: Aug. 30, 2022
(86) PCT No.: PCT/US2022/041960

§ 371 Date: Feb. 21, 2024

Related U.S. Application Data
(60) Provisional application No. 63/240,566, filed on Sep. 3, 2021.
(60) Provisional application No. 63/246,326, filed on Sep. 21, 2021.

(51) Int. Cl. A61F 5/01

(52) U.S. Cl. CPC A61F 5/0127; A61F 2005/0155

(57) ABSTRACT

A device that may be wearable is configurable to dynamically cycle through multiple states. The device may include a first member extending from a first end to a second end and a second member extending from a first end to a second end with the first member rotatably connected to the second member about a first axis. A drive mechanism is included for driving relative rotation between the first member and second member about the first axis. Additionally, a system may be employed for initiating the dynamic cycling between two states. The device and system may be used to treat an individual or animal.

### CROSS-REFERENCE TO RELATED APPLICATIONS

This application claims priority to U.S. Provisional Patent Application No. 63/240,566, filed Sep. 3, 2021, and U.S. Provisional Patent Application No. 63/246,326, filed Sep. 21, 2021, the entire contents of which are incorporated herein by reference.

### BACKGROUND

The disclosed embodiments relate to wearable devices, more particularly a wearable splint, and even more particularly a dynamic splint for manipulating a user's body parts around a joint. The disclosure also relates to a related method of dynamically manipulating a body part and a system therefor that utilizes an input device in communication with the wearable device.

Plantar Fasciitis is a common condition affecting the feet, and is associated with damage to the connective tissue which supports the arch of the foot. A common treatment device for plantar fasciitis is a night splint that is used to relieve pain from plantar fasciitis. Night splints are generally braces that attach to the foot and lower leg to hold the foot in fixed position at night while the user sleeps. The objective of night splints is to treat the user via stretching the plantar fascia ligament throughout the night.

Traditionally, night splints maintain the lower leg in a fixed dorsiflexed position, with toes maintained in an upward position. A common drawback with night splint is that they are relatively uncomfortable to wear, especially over long periods of time and/or for the multiple nights of consecutive wear, which are both necessary to realize a clinical benefit. Discomfort typically leads to poor treatment compliance by users. Further, although night splints stretch the plantar fascia ligament, the static nature of the stretch does not closely mimic the motion of the lower leg during a user's waking hours, wherein the foot shifts from dorsiflexion (toes upward) to plantar flexion (toes downward).

No wearable devices exist that provide for movement of a user's foot in this manner, cycling between dorsiflexion and plantar flexion. It would thus be useful to provide a device, system and method for dynamic stretching during use.

### SUMMARY

One embodiment provides a wearable device, such as an orthopedic splint, that cycles the foot between dorsiflexion and plantar flexion. This dynamic motion can be used to stretch the plantar fascia and other anatomic structures.

In one embodiment, a wearable article comprises a first member, a second member and a drive mechanism. The first member extends from a first end to a second end. The second member extends from a first end to a second end and is rotatably connected to the first member about a first axis toward their respective first ends. The drive mechanism drives relative rotation between the first member and second member about the first axis.

In another embodiment, a system for initiating dynamic cycling between two states in a wearable device is disclosed. The system includes a wearable device configured to dynamically cycle between a first state and a different second state. A data input and transmission device is configured to receive and optionally store data input from a user. A data receiving module is in communicative contact with the data input and transmission device and is configured to receive and optionally store data from the input device. The data receiving module is configured to initiate or drive dynamic cycling between the two states of the wearable device in response to the data input into the input device.

Also disclosed is a use of a device for treating an individual or animal. A device configured to dynamically cycle between a first state and a different second state is provided, and the device is engaged to a portion of a body part of the individual or animal. A program whereby the device cycles between the first state and the second state at predetermined intervals and optionally maintained in each of said first state and second state for predetermined durations is then initiated.

In certain embodiments and treatment regimes, the device is a splint that is configured to be worn at night and cycles while the wearer is asleep.

In one embodiment, the device dynamically cycles the lower foot through a range of motion including dorsiflexion and plantar flexion.

In another embodiment, the device cycles the lower foot from a neutral position, to dorsiflexion, and back to a neutral position.

In another embodiment, the device cycles the lower foot through a range of motion including dorsiflexion, plantar flexion and one or more intermediate positions between dorsiflexion and plantar flexion.

The disclosed embodiments achieve dynamic stretching of the plantar fascia ligament and associated structures.

The device is preferably configured to provide a more gradual rate of cycling and applied force, such as to assist in not waking the user.

In one embodiment the device is equipped with a sensor that initiates the dynamic cyclic motion when the user is determined to be asleep.

The device may include one or more of a plurality of different mechanisms or units for providing the cyclic motion, including linear actuators, lead screw mechanisms, hydraulic mechanisms, and spooled cable drive mechanisms, for example.

Notably, while the disclosure and depicted embodiments are presented primarily within the context of a foot splint for treatment of plantar fasciitis, the inventiveness of the embodiments are in no way limited as such. Other embodiments exist that may be worn in other areas to dynamically move other body parts, such as a knee, elbow or finger, for example. The terms “splint” and/or “night splint” shall be interpreted in their broadest sense and be synonymous with “wearable device.” Likewise, all reference to a human user's foot, leg and/or toes is exemplary in nature and shall apply to any other body part that is movable in a similar cyclical manner and to applicable non-human animals.

### BRIEF DESCRIPTION OF THE DRAWINGS

FIGS. 1A-1C show exemplary night splints as known in the prior art;

FIG. 2 depicts a foot and shows dorsiflexion and plantar flexion positions for reference;

FIGS. 3A and 3B show another general representative embodiment of a night splint as known in the prior art;

FIGS. 4A and 4B show a representative embodiment of the disclosed dynamic splint;

FIG. 5 shows the splint of FIGS. 4A and 4B with a cable shield;

FIG. 6 shows perspective views of a preferred embodiment of the disclosed dynamic splint with certain elements removed for clarity;

FIG. 7 shows elevation views of the splint of FIG. 7;

FIG. 8 shows a bottom view of the bottom foot member of the splint showing a gear and cord actuation mechanism;

FIG. 9 shows another bottom perspective and bottom elevation view of the dynamic splint;

FIG. 10 show additional views of the dynamic splint;

FIG. 11 depicts an alternate drive assembly for use with the disclosed embodiments of the dynamic splint;

FIG. 12 shows photographs of an example of another embodiment of the dynamic splint configured for engagement at the front of the leg and top of the foot;

FIG. 13 shows photographs of an example of the embodiment of FIG. 6;

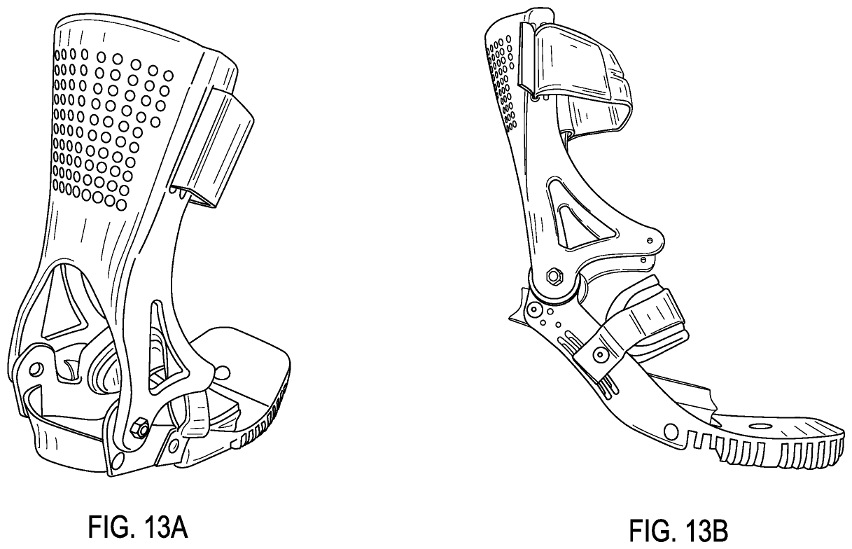

FIG. 14 shows another preferred embodiment of the disclosed dynamic splint; and

FIG. 15 shows bottom views of the embodiment of FIG. 14.

### DETAILED DISCLOSURE

Among the benefits and improvements disclosed herein, other objects and advantages of the disclosed embodiments will become apparent from the following wherein like numerals represent like parts throughout the figures. Detailed embodiments of a wearable device and dynamic splint, system for initiating dynamic cycling between two states within the wearable device, and a use thereof, are disclosed; however, it is to be understood that the disclosed embodiments are merely illustrative of the invention that may be embodied in various forms. In addition, each of the examples given in connection with the various embodiments of the invention are intended to be illustrative, and not restrictive.

Throughout the specification and claims, the following terms take the meanings explicitly associated herein, unless the context clearly dictates otherwise. The phrase “in some embodiments” as used herein does not necessarily refer to the same embodiment(s), although it may. The phrases “in another embodiment” and “in some other embodiments” as used herein do not necessarily refer to a different embodiment, although it may. Thus, as described below, various embodiments may be readily combined without departing from the scope or spirit of the invention.

In addition, as used herein, the term “or” is equivalent to the term “and/or,” unless the context clearly dictates otherwise. The term “based on” is not exclusive and allows for being based on additional factors not described unless the context clearly dictates otherwise. In addition, throughout the specification, the meaning of “a,” “an,” and “the” include plural references. The meaning of “in” includes “in” and “on.”

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

With reference to the drawings, disclosed herein is a dynamic splint 10 that most generally includes a bottom (foot/boot) member 12 rotatably engaged with a top (leg) member 14. In the depicted preferred embodiments, a cord or cable 18 extends between and is attached to each of the bottom member 12 and top member 14 at a position forward (in the bottom member) and above (in the top member) the respective heel ends with a rotation/pivot axis 16 toward the heel.

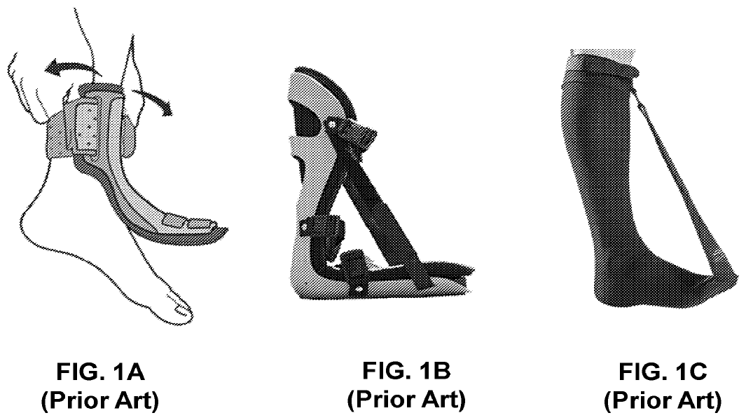

FIG. 1 depicts various exemplary prior art static night splints. The night splint of FIG. 1A comprises a relatively rigid component configured to be attachable to the front of the lower leg and top of the foot. Straps wrap around the back of the lower leg and the bottom of the foot to maintain the position of the splint and force the lower leg and foot into a desired position. The relative angle between the lower leg and foot is substantially defined by the shape of the substantially rigid portion of the device. There is little to no adjustability and no cycling capability.

The night splint FIG. 1B comprises a rigid component fit to the back of the lower leg and the bottom of the foot. Straps hold the lower leg and foot securely within the device and adjustment straps extend from the lower leg region to the foot region. The adjustment straps can be adjusted to change the relative angle between the lower leg and foot, but once set, the degree of dorsiflexion or plantar flexion is fixed for the duration of use.

The night splint of FIG. 1C includes an adjustable strap extending from a user's leg to the toe region and may be incorporated into a sock or similar article. Like the prior art splints of FIGS. 1A and 1B, the splint of FIG. 1C only provides static treatment. Further, the flexing and/or stretching provided by this type of splint does not specifically target the plantar fascia, instead targeting multiple forefoot structures and areas.

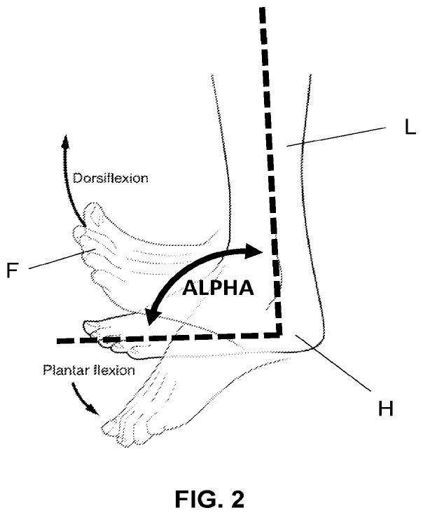

FIG. 2 depicts dorsiflexion and plantar flexion of the foot relative to a neutral position, typically defined as the foot being substantially perpendicular to the lower leg. The foot typically naturally takes a plantar flexed position when a person is asleep. Night splints typically acts to maintain approximately 10-degrees of dorsiflexion. Throughout this disclosure references are made to the position of the foot. These descriptions are not intended to limit the scope of future claims, but rather to describe relative positions and motions. For example, if an embodiment is described as moving the foot from a neutral position into dorsiflexion, one of skill in the art would readily understand that the device may also be configured to move the foot from plantar flexion into dorsiflexion.

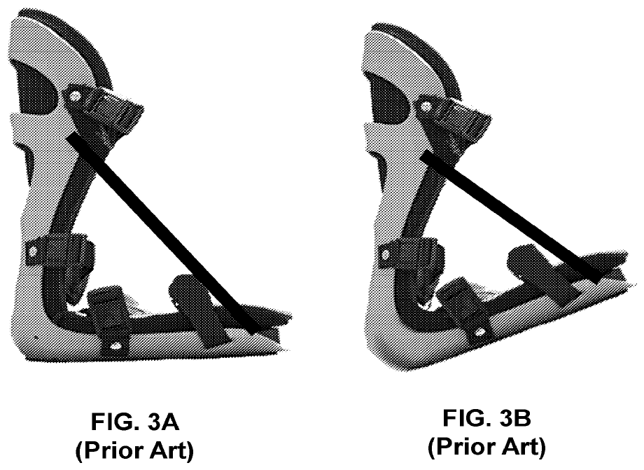

FIGS. 3A and 3B depict an embodiment of a night splint configured to be attached to the posterior (rear) side of the lower leg and plantar (bottom) side of the foot. A tensioner, which includes one or more cables attaches to an upper (distal) region of the upright leg member and front (distal) region of the lateral foot member and sets the splint angle α as defined by the length of the cable(s). The angular relationship between lower leg and foot is defined by splint angle α (see FIG. 2). As the tensioner/cable length increases, the angle α increases, and vice versa.

In use, the user secures the night splint with the leg and foot straps. The night splint then dynamically changes tensioner length to initiate a change in the angle α. Shortening tensioner length causes the foot to move toward or into dorsiflexion, while increasing tensioner length allows the foot to move toward or into its natural plantar flexion position. In operation, the representative splint cycles through shorter and longer tensioner lengths at predetermined periods of time in order to cycle the user's foot between dorsiflexion and planar flexion.

In certain embodiments the cycle time can be in the order of seconds or minutes. In certain embodiments, the cycle program is set to maintain the foot at one or more intermediate fixed positions between dorsiflexion (minimum cable length) and plantar flexion (maximum cable length) for seconds or minutes. In another embodiment, cable/tensioner length is reduced from a maximum length, plantar flexion position to a dorsiflexion position over a period of one minute; cable/tensioner length is then maintained at the minimum length for 10 minutes; then cable/tensioner length is lengthened to a maximum length over a period of one minute; the new tensioner length L is then maintained for 10 minutes, and the cycle is repeated through the duration of treatment. These representative durations are non-limiting.

Changes to tensioner length can be implemented via a variety of mechanisms, including linear actuators, for example. Specific linear actuator types include electro-mechanical actuators, hydraulic actuators and pneumatic actuators among others.

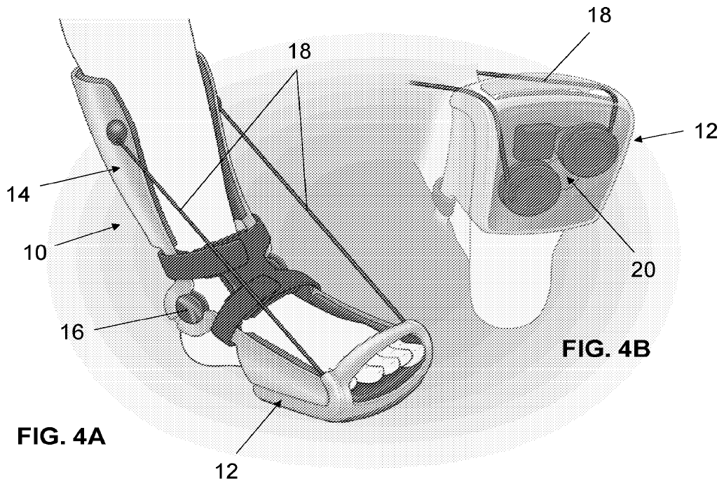

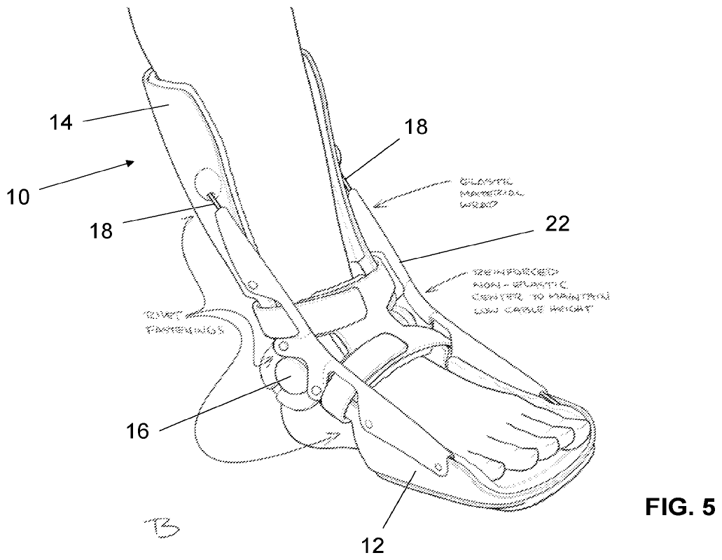

FIGS. 4A-5 depict another exemplary dynamic splint comprising an upper leg member 14 rotatably engaged with a lower boot member 12 at the hinge axis 16. A cable or pair of cables 18 are communicatively engaged with a drive system 20 disposed on the bottom of the lower member 12. The gear(s) of the drive system are operated by an integral motor to initiate rotation in opposite directions to shorten the cables 18 to initiate dorsiflexion and allow lengthening of the cables 18 to initiate plantar flexion. The splint 10 may be attached to the user's foot via one or more straps, the depicted position of which is non-limiting. Notably, the rear section of the splint 10 at the bottom end of the top member 14 and rear end of the lower member 12 is opened to allow the user's heel to be free. Some embodiments include an additional heel strap to hold the splint in place.

FIG. 5 depicts a cable shield 22 connected to the splint on each side to hold each cable in a low profile position.

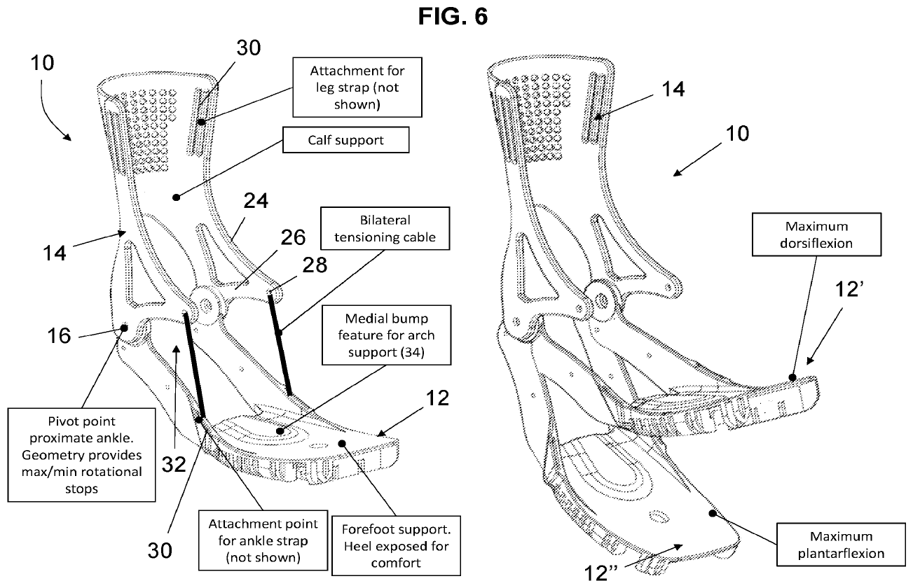

FIG. 6 shows a preferred embodiment of the disclosed dynamic splint 10, including an upper leg member 14 rotatably engaged within a lower foot member 12 at an axis 16. The splint 10 is sized and shaped for the axis 16 to be proximate a user's ankle when worn for comfort and to provide the greatest possible range of angles during use. As shown, the upper member 14 includes a cooperative pair of arms 24 and 26 that extend forward therefrom in a cantilevered manner to a cable attachment end 28. This cantilevered position allows use of shorter cable sections to provide the full range of motion, which in turn allows a more constant cable tension and smoothness over the full range.

As shown, the lower and upper members 12 and 14 include a plurality of slots or openings 30 for use in attaching straps to attach the members to a user's leg L and foot F. While not depicted, one can readily understand that straps can engage via the slots 30 in the upper member 14 and in the lower member 12 configured to wrap around the front of a user's lower leg and over the top of the user's foot, respectively. Also not depicted, in some embodiments, one or more rear straps extend between the right and left side of one or both of the upper member 14 and lower member 12 proximate the heel or Achilles position for assisting attachment and preventing the user's heel from moving too far rearward out from the rear opening 32. Holes may additionally be defined within one or more of the upper and lower members for ventilation.

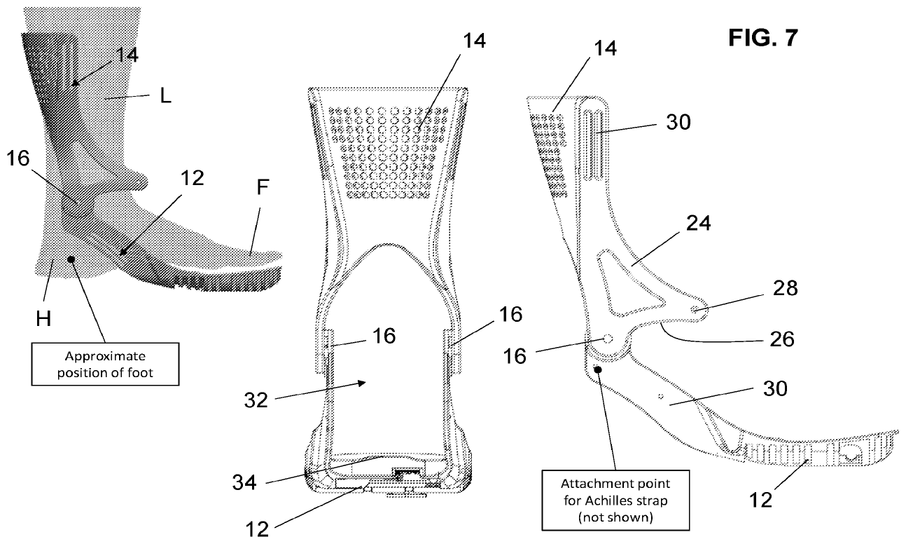

FIG. 7 shows different elevation views of the split of FIG. 6, including a side elevation view in place on a representative foot F and leg L. Various straps are again hidden for clarity. The right-most view clearly depicts the forward position of the cantilevered arms 24 and 26 collectively defining the cable attachment end 28. Notably, embodiments exist with only a single arm or even more than two arms, however the dual arm quasi-triangular configuration has been shown to be especially robust.

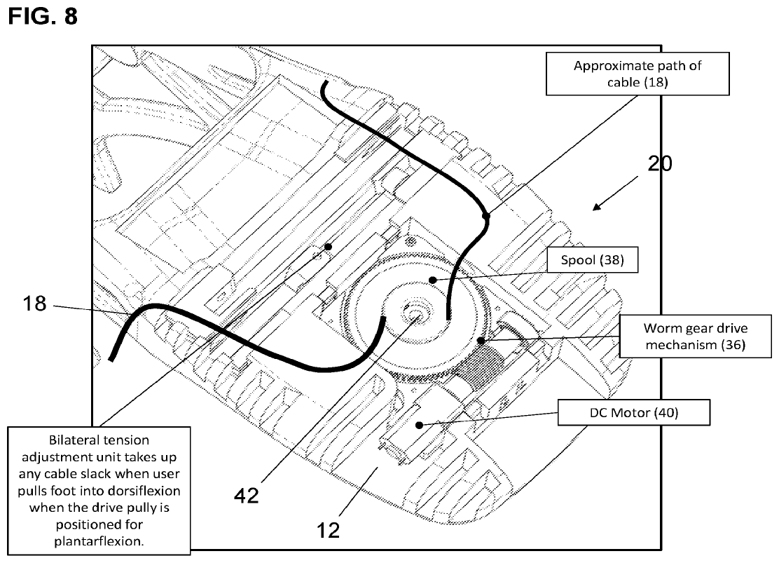

FIG. 8 shows an enlarged view of the bottom of the bottom foot member 12 to show details of an embodiment of a drive mechanism 20. As shown, the drive mechanism 20 includes a worm gear 36 with threading engaged with teeth of a gear spool 38. The cable 18 runs through the central axis of the spool 38 from diametrically opposite sides. Alternatively, respective ends of the cables 18 are secured to the spool 38 at substantially diametrically opposite positions. A DC motor 40 is operatively associated with the worm gear 36. As one can realize, the motor drives the worm gear in a first direction to initiate rotation of the spool 38 about the axis 42 in a first direction to shorten the cables 18 and bring the splint 10 into dorsiflexion position. From a dorsiflexion position, the motor can reverse and drive the spool 38 in an opposite second direction to initiate rotation of the spool 38 about the axis 42 in an opposite second direction to allow the cable sections 18 to lengthen and relax tension and allow the splint 10 to move toward a plantar flexion position. While also not depicted, the bottom member 12 may define one or more cable channels for the respective cables 18 to pass through internally so that they are not exposed. While it may be described as two separate cables 18, a preferred embodiment includes a single cable 18 that passes diametrically through axis 42 of the spool 38 and is wrapped around or unwrapped from a central portion when the spool rotates. Additionally, not depicted is a power source, such as a battery, for powering the motor.

Finally, preferred embodiments exist with a data receiving unit, which may include writable storage, such as a processor for receiving data input by a user and executing programable steps. Understandably, the position of the drive mechanism 20 and associated elements can be in the top leg 14 instead of the bottom member 12.

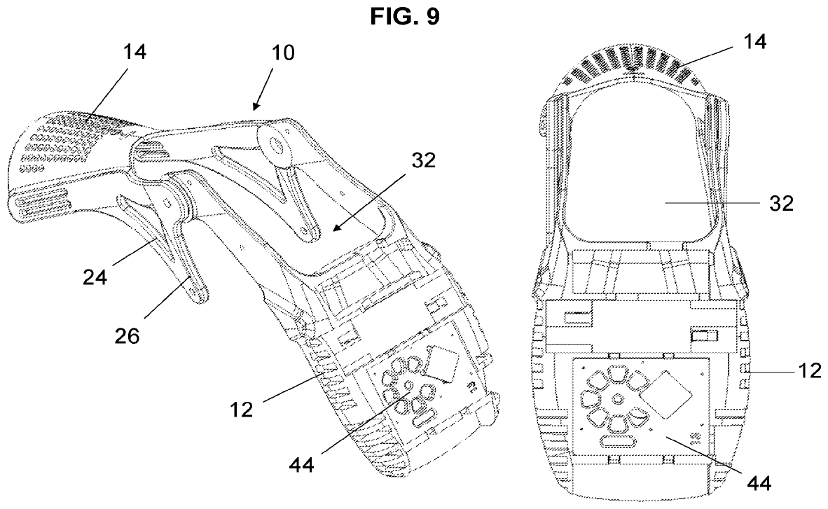

FIG. 9 shows two bottom views of the splint 10, and specifically shows an exemplary shield 44 in position to conceal the drive mechanism 20. The shield 44 is preferably removable to allow access to the drive mechanism.

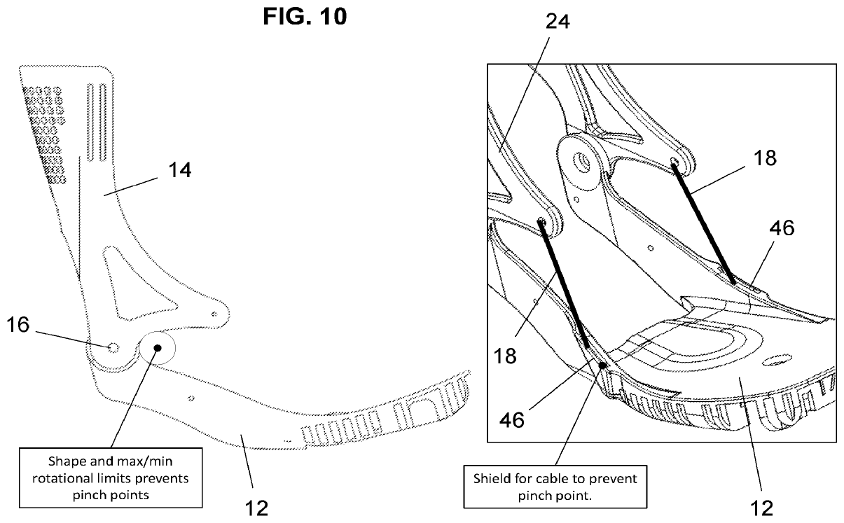

FIG. 10 shows additional views of the splint 10. Representative cables 18 are shown in the enlarged view on the right side. As shown, the cables 18 may extend from the bottom member 12 through slots or openings 46. As shown, the configuration including the cantilevered position of the cable attachment point 28 provides a shortened length of travel for the cable 18, which helps keep tension in the cables substantially constant over a full range of motion between minimum length (dorsiflexion) and maximum length (plantar flexion). In one embodiment, a constant force spring may be communicatively engaged with the spool to help ensure smooth extension and retraction of the cables.

In this embodiment, the upper surface of the lower member 12 has a contour configured for improved wearability and user comfort, including for example a medial bump 34 for providing arch support. In one embodiment, the medial bump 34 is shiftable or indexable to accommodate either a left foot or a right foot, such that a single splint may be worn on either foot/leg. The right side of FIG. 6 depicts the splint with the bottom member 12 in a neutral position (cables at an intermediate length), maximum dorsiflexion position 12′ (cables 18 at minimum length) and maximum plantar flexion position 12″ (cables 18 at maximum length).

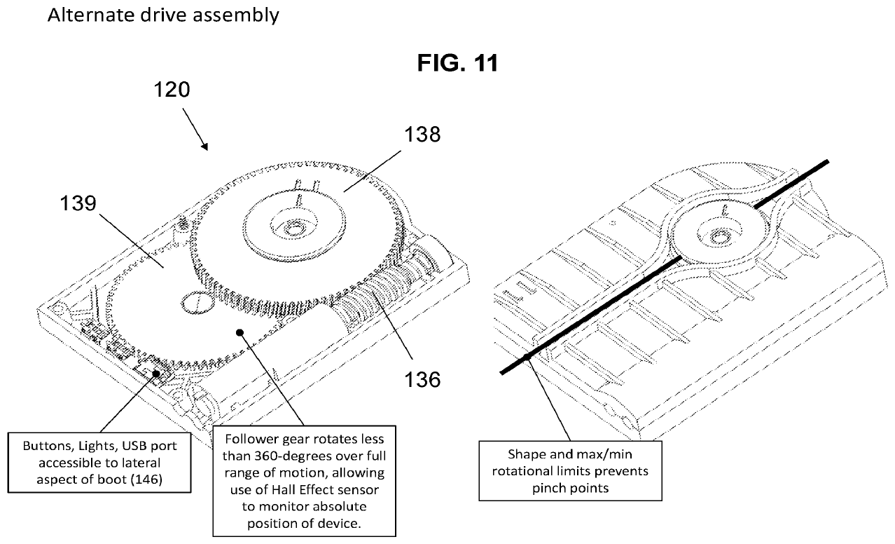

FIG. 11 shows an alternative embodiment of a drive assembly 120 that is modular and configured to be installed and optionally uninstalled in a portion of the splint 10. Functionally, the drive assembly 120 is substantially similar to the integral assembly 20 shown in FIG. 8, and includes a spool 138 driven by a worm gear 136 that is powered by a motor. As shown, the cable 18 extends diametrically through the spool 138. A power source (battery) can be integral to the module 120 or can be present on the splint and configured to communicatively connect to the module when installed. Also shown in FIG. 11 are optional elements such as buttons, lights or a USB or similar connection port.

The exact configuration of the drive mechanism is merely exemplary and non-limiting. For example, different types or positions of motors can be employed; the spool can be in different positions relative to the motor or worm gear; different types of drive mechanisms can be employed.

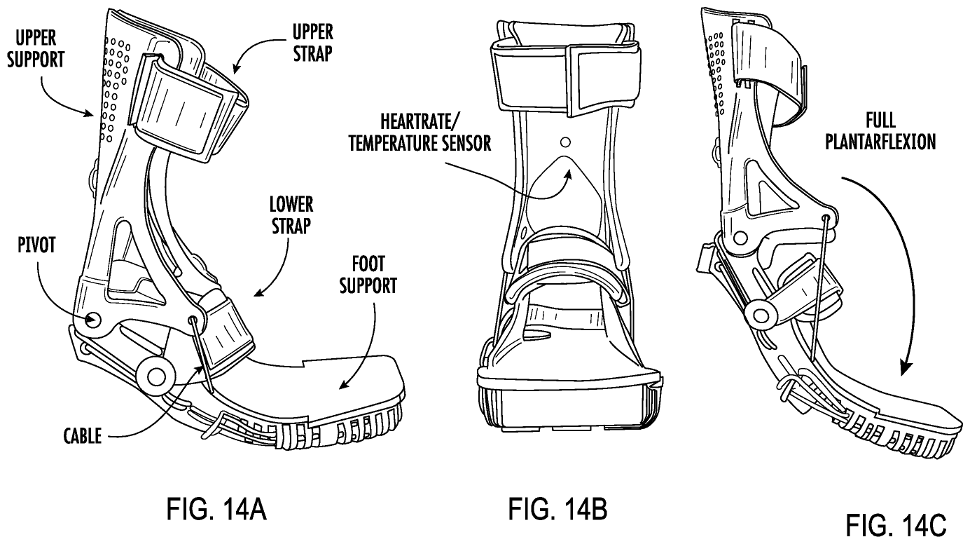

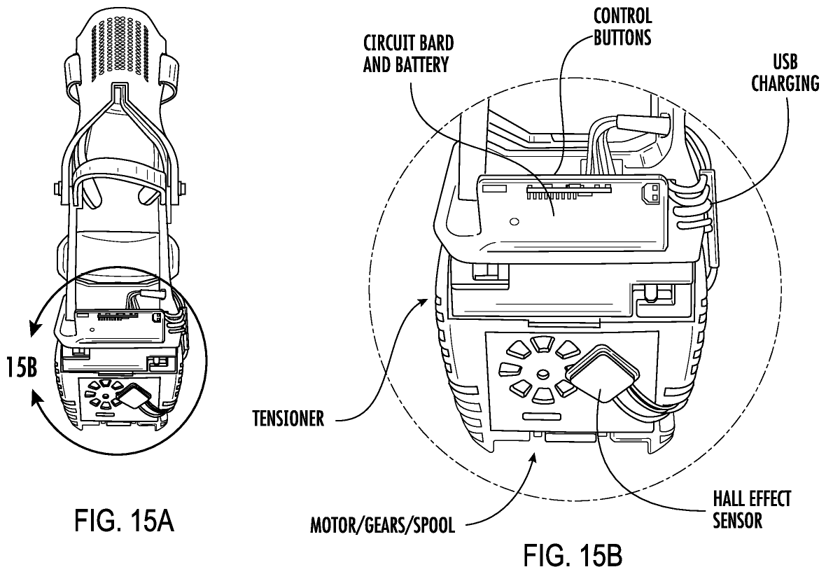

FIGS. 14 and 15 show views of another closely related embodiment of the dynamic splint. As shown in FIG. 14, this embodiment includes heart rate and temperature sensors mounted on the inner portion of the top member in an area that will allow contact with the skin proximate the calf muscle of a wearer. Also shown are various straps for attachment to the individual and the cable extending between the top member and bottom member. As shown in FIG. 15, included within the bottom member are a charging port, battery, circuitry, Hall effect sensor and the drive mechanism.

Various alternative embodiments or configurations from those described above exist, for example:
A shield or similar element may span all or a portion of tensioner/cable length between the top member and bottom member, which may be substantially cylindrical in shape.
The actuator may provide tension in a direction to induce dorsiflexion while in an active state, and negligible tension when in an inactive state, allowing the foot to take a natural position (plantar flexion).
The range of motion (i.e. limits on tensioner/cable length) may be force-controlled instead of distance/displacement-controlled.
Range of motion may be limited by mechanical stops which can be set prior to operation of the device.
The range of motion may alternatively or additionally be limited by an input parameter within software.
A single tensioner or multiple tensioners may be located on the medial and/or lateral position(s) of the splint. In embodiments that utilize multiple actuators, are used, an additional degree of freedom may be controlled, which can carry additional therapeutic benefits.
An ankle and/or foot strap proximate the axis of rotation to promote constant strap force across the top of foot across the full range of motion.
An ankle and/or foot strap used in conjunction with the Achilles strap to prevent device from sliding off under full plantar flex.
One or more specific geometric configurations of the arm to maintain a preferred minimal distance between the cable path and the axis of rotation across the full range of motion.
Multiple rotational axes (intermediate link) or irregular cam for providing improved biomechanics, resulting in a “shifting” rotational axis at different points of actuation.
Cables that exit opposite sides of the spool to assist in balancing forces within the drive assembly, resulting in less friction and thereby providing greater mechanical efficiency.

Further, alternate device configurations could achieve a change in foot angle α other than by a linear actuator. For example, a spring located near the device's axis of rotation (proximate the ankle) could provide a force to nominally move the foot into a dorsiflexed position. In this alternative embodiment, a cable running behind the foot and lower leg could be tightened to pull the device into a neutral or plantar flexed position. A similar cable configuration, in which the cable is wound (tightened) using an electric motor, can be used to change the length of a tensioner on the front/top of the anatomy.

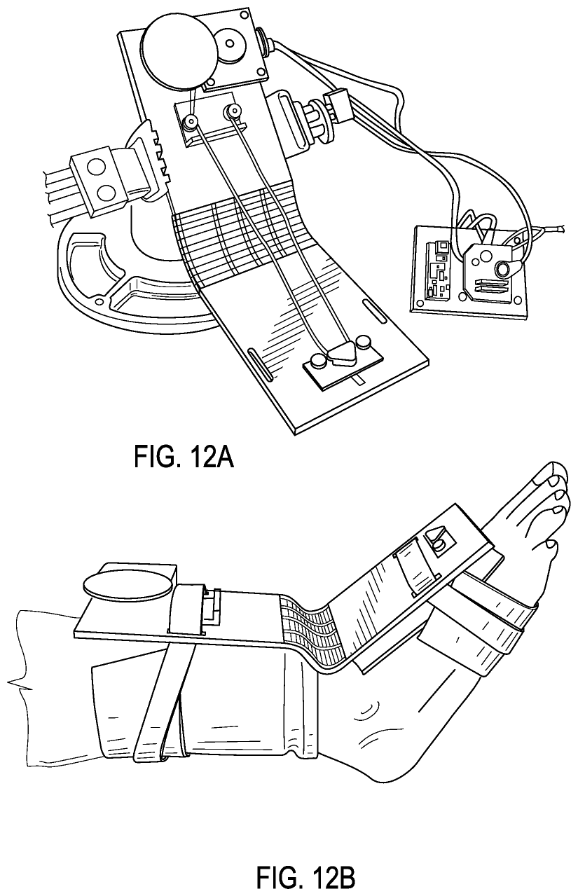

In another embodiment shown generally in FIG. 12, the splint is configured for attachment to a user's lower leg and foot from the anterior (i.e., front) and dorsal (i.e., top) surfaces, respectively. Like the previously disclosed embodiments, straps are employed to secure the lower leg and foot into contact with the rigid portions of the device. A tensioner with cables extends between the leg portion of the device and the foot portion of the device.

The angle α and cable length are directly related, i.e., as the length of the cables increases, the angle α also increases. In use, the user secures the night splint to the leg and foot using the appropriate straps. The night splint then dynamically changes tensioner length L to alter the angle α. Shortening cable length causes the foot to move toward or into dorsiflexion. Increasing cable length causes the foot to move toward or into plantar flexion. Like the other embodiments, in operation the night splint cycles through shorter and longer tensioner lengths in a treatment sequence that may be programmed.

Embodiments may incorporate one or more sensors to determine when the user is asleep, and initiating activation of the device only when the user is determined to be asleep. For example:
Sleep state may be determined by a sensor that monitors one or more of motion, sound, heartrate and respiratory rate.
Device motion may be stopped when the device senses that the user is awake or waking.
Sensors may be integrated into the night splint or accessible through communication with a separate removable sensor located on in proximity to the user. Communication may be via wireless technology.

In certain embodiments, a user interface and input device may be a wired display interface or a mobile app on a phone or tablet, for example, that communicated wirelessly with a module in the splint. The software allows users to control parameters, including cycling rate, position hold times, angular distance of the motion, duration of active treatment, among others. The app may also allow monitoring of device use and includes features to encourage compliance, including graphical display of use, reminders, scheduling of future treatments, assessments of applied force and range of motion, and user ratings for symptom pain.

As noted above, the invention is not limited specifically to a night splint, foot region or treatment of plantar fasciitis. Alternate embodiments exist for treating other anatomic sites and conditions in which application of dynamic motion has a therapeutic benefit. For example, a dynamic brace or splint exists configured for cycling a knee, wrist, elbow or finger. Moreover, the dynamic operation of the disclosed device can provide an additional benefit by increasing circulation in the area of the body, which carries therapeutic benefits beyond plantar fasciitis specifically. Further, the treatment method is not limited to dynamic motion about a joint. Embodiments of the method of treatment exist for applying any other type of therapy that alternates between two states while a wearer sleeps. For example, an embodiment exists that utilizes for cyclically applying compression to a limb. Such an embodiment may employ an input device, such as a mobile app, to communicate data to the device. Other non-limiting examples include embodiments of the device that initiate cycling of different pressures on a body part, different temperatures, and different elevation states or levels.

While a preferred embodiment has been set forth for purposes of illustration, the foregoing description should not be deemed a limitation of the invention herein. Accordingly, various modifications, adaptations and alternatives may occur to one skilled in the art without departing from the spirit of the invention and scope of the claimed coverage.

1. A wearable article, comprising:
a first member extending from a first end to a second end;
a second member extending from a first end to a second end and rotatably connected to the first member about a first axis toward their respective first ends; and
a drive mechanism for driving relative rotation between the first member and second member about the first axis.

2. The wearable article of claim 1, comprising one or more cords extending between and attached to each of the first member and second member between their respective first and second ends.

3. The wearable article of claim 2, wherein the one or more cords is operatively engaged with the drive mechanism such that the drive mechanism initiates the relative rotation via one or both of contracting and lengthening a section of the one or more cords that extends between the first member and second member.

4. The wearable article of 3, wherein one or both of the first member and second member includes an arm extending outward and in a direction toward the other respective member and the cord is attached to the arm.

5. The wearable article of claim 1, comprising one or more straps for attaching the article to an individual or animal to initiate movement at a joint thereof.

6. The wearable article of claim 1, wherein the article is configured to be worn by an individual or animal on the foot and leg.

7. The wearable article of claim 1, wherein the drive mechanism initiates a program of relative rotation in opposite directions.

8. The wearable article of claim 7, wherein the drive mechanism optionally stops and maintains the first member and second member in one or more different relative rotational positions during execution of the program.

9. The wearable article of claim 1, comprising an opening between the first member and second member in an area proximate the axis.

10. The wearable article of claim 1, wherein the drive system is programable to initiate multiple different predetermined programs of rotation.

11. The wearable article of claim 1, wherein the first member and second member extend obliquely relative to one another at a first intermediate angle and the drive mechanism (a) initiates relative rotation to a second minimum angle smaller than the first angle, and (b) allows rotation to a third maximum angle greater than the first angle.

12. The wearable article of claim 11, wherein the drive mechanism is configured to stop at multiple different angles between the second minimum angle and third maximum angle.

13. A system for initiating dynamic cycling between two states in a wearable device, the system comprising:
a wearable device configured to dynamically cycle between a first state and a different second state;
a data input and transmission device configured to receive and optionally store data input from a user; and
a data receiving module in communicative contact with the data input and transmission device configured to receive and optionally store data from the input device, wherein
the data receiving module is configured to initiate or drive dynamic cycling between the two states of the wearable device in response to the data input into the input device.

14. The system of claim 13, wherein the wearable device comprises a first member and a second member rotatably attached to the first member, and the data receiving module initiates or drives relative rotation between the first member and second member about a first axis.

15. The system of claim 13, wherein the data input and transmission device is a mobile device running an application or other software.

16. The system of claim 13, wherein the wearable device is the device comprises:
a first member extending from a first end to a second end;
a second member extending from a first end to a second end and rotatably connected to the first member about a first axis toward their respective first ends; and
a drive mechanism for driving relative rotation between the first member and second member about the first axis.

17. The system of claim 13, comprising a sensor for determining when the user is in a sleeping state and thereafter initiates the program of cycling between the two states.

18. The system of claim 13, comprising software or an application for tracking and optionally storing data including use, parameters of two or more states, start time, stop time, duration of steps, schedules, symptoms, opinions, quality of sleep, and a unit of force or units of forces required to achieve a stretching target.

19. The system of claim 18, wherein the software or application allows a user to input one or more of the data points.

20. Use of a device for treating an individual or animal, comprising:
providing a device configured to dynamically cycle between a first state and a different second state;
engaging the device to a portion of a body part of the individual or animal; and
initiating a program whereby the device cycles between the first state and the second state at predetermined intervals and optionally maintained in each of said first state and second state for predetermined durations.
